(19) United States
(12) Patent Application Publication
(10) Pub. No.: US 2025/0137012 A1
(43) Pub. Date: May 1, 2025

(54) COMPOSITIONS AND METHODS OF USING TWO-PROMOTER VECTOR FOR TREATMENT OF LYSOSOMAL STORAGE DISORDERS

(71) Applicant: M6P THERAPEUTICS, INC., St. Louis, MO (US)

(72) Inventors: Hung DO, Edmond, OK (US); Lin LIU, Ballwin, MO (US); Andrew Charles HEDMAN, St. Louis, MO (US)

(21) Appl. No.: 18/835,310

(22) PCT Filed: Jan. 26, 2023
(86) PCT No.: PCT/US2023/011617

§ 371 Date: Aug. 1, 2024

(51) Int. Cl. C12N 15/86; A61K 35/76; A61K 38/45; A61K 38/47; A61K 48/00; C12N 9/12; C12N 9/24

(52) U.S. Cl. CPC C12N 15/86; A61K 35/76; A61K 38/45; A61K 38/47; A61K 48/005; C12N 9/1288; C12N 9/2402; C12N 2750/14143; C12N 2830/205; C12Y 207/08017; C12Y 302/01022; C12Y 302/01024; C12Y 302/01045; C12Y 302/01046; C12Y 302/0105; C12Y 302/01052

(57) ABSTRACT

Provided are compositions comprising vectors for the co-expression of a modified GlcNAc-1-Phosphotransferase gene and a lysosomal enzyme. The gene encoding the lysosomal enzyme is operably linked to a first promoter and the gene encoding the GlcNAc-1-Phosphotransferase is operably linked to a second promoter. Also provided herein are methods of treating a lysosomal storage disorder comprising administering to a subject the compositions of the disclosure.

### CROSS REFERENCE TO RELATED APPLICATIONS

This application is the U.S. national stage filing of International Application No. PCT/US2023/11617, which itself claims the benefit of priority to U.S. Patent Application Ser. No. 63/306,615, filed Feb. 4, 2022, the entire contents of which are incorporated herein by reference.

### BACKGROUND

Lysosomal storage disorders (LSDs) relate to inherited metabolic disorders that result from defects in lysosomal function. Currently, about 50 distinct LSDs have been identified but a small number of these (fewer than 10) are reported to have treatments. Therefore, there is an unmet need in the art for safe and effective treatments for LSDs. The disclosure provides two solutions for this unmet need, through either enzyme replacement therapy (ERT) or gene therapy.

### SUMMARY

The disclosure provides a composition comprising a vector comprising a sequence encoding a first promoter operably linked to a first polynucleotide encoding a lysosomal enzyme and a second promoter operably linked to a second polynucleotide encoding a modified GlcNAc-1 phosphotransferase (PTase).

In some embodiments, the vector of the disclosure is a viral or non-viral vector. In some embodiments, the vector of the disclosure is an adenoviral vector or an adeno-associated viral (AAV) vector or a plasmid.

In some embodiments, the first promoter of the vector of the disclosure is CBH and the second promoter is selected from EFS or JeT.

In some embodiments, the modified GlcNAc-1 phosphotransferase of the vector of the disclosure comprises S1S3 PTase.

In some embodiments, the lysosomal enzyme of the vector of the disclosure is selected from the group consisting of b-glucocebrosidase (GBA), Galactosylceremidase (GALC), a-Galactosidase (GLA), a-N-acetylglucosaminidase (NAGLU), acid a-glucosidase (GAA), b-hexosaminidase (HexM) and lysosomal acid a-mannosidase (LAMAN).

In any of the foregoing embodiments, the vector of the disclosure further comprises a 3′ UTR. In some embodiments, the 3′ UTR is selected from SV40 and bGH poly-A.

In any of the foregoing embodiments, the composition of the disclosure further comprises a pharmaceutically acceptable carrier.

The disclosure also provides a method of treating a lysosomal storage disorder (LSD), the method comprising administering to a subject an effective amount of any of the foregoing compositions, wherein the composition increases the phosphorylation of a lysosomal enzyme responsible of the LSD, thereby treating the LSD.

In some embodiments, the subject has been diagnosed with the LSD and/or the subject presents a sign or symptom of the LSD.

The disclosure also provides a method of preventing an occurrence or an onset of a lysosomal storage disorder (LSD), the method comprising administering to a subject an effective amount of a composition of any of the foregoing compositions, wherein the composition increases the phosphorylation of a lysosomal enzyme responsible of the LSD, thereby preventing the occurrence of the LSD in the subject.

In some embodiments, the subject is at risk of the occurrence or the onset of the LSD and/or the subject presents a sign or a symptom of the LSD.

The disclosure also provides a method of ameliorating the phosphorylation of a lysosomal enzyme responsible for a lysosomal storage disorder (LSD), the method comprising contacting to a cell, an effective amount of any of the foregoing compositions, wherein the composition increases the phosphorylation of the lysosomal enzyme.

In some embodiments, the cell is in vitro or ex vivo.

In some embodiments, a subject comprises the cell. In some embodiments, the subject presents a sign or a symptom of the LSD, the subject is at risk of the occurrence or the onset of the LSD, and/or the subject has been diagnosed with the LSD. In any of the foregoing embodiments, the subject is a human.

In any of the foregoing methods of the disclosure, the administering comprises a systemic route of administration or a local route of administration. In some embodiments, the systemic route of administration is enteral, parenteral, oral, intramuscular (IM), subcutaneous (SC), intravenous (IV), intra-arterial (IA), intrathecal, intraspinal, or intraventricular.

### BRIEF DESCRIPTION OF THE DRAWINGS

For the purpose of illustrating the disclosure, there are depicted in the drawings certain embodiments of the disclosure. However, the disclosure is not limited to the precise arrangements and instrumentalities of the embodiments depicted in the drawings.

FIG. 1 is a graphical depiction of the two-promoter gene expression cassettes for expression of S1S3 PTase and GLA, including inside-out, outside-in and sequential configurations.

FIG. 2A is a graphical depiction of the two-promoter gene expression cassettes for expression of S1S3 PTase and GLA in the inside-out configuration transfected into HEK 293T cells.

FIG. 2B depicts GLA activity in lysates of the HEK 293T cells transfected with gene expression cassettes in the inside-out configuration.

FIG. 2C depicts a Western Blot showing GLA, S1S3 PTase, and GAPDH protein in lysates of the HEK 293T cells transfected with gene expression cassettes in the inside-out configuration.

FIG. 3A is a graphical depiction of the two-promoter gene expression cassettes for expression of S1S3 PTase and GLA in the outside-in configuration transfected into HEK 293T cells.

FIG. 3B depicts GLA activity in lysates of the HEK 293T cells transfected with gene expression cassettes in the outside-in configuration.

FIG. 3C depicts a Western Blot showing GLA, S1S3 PTase, and GAPDH protein in lysates of the HEK 293T cells transfected with gene expression cassettes in the outside-in configuration.

FIG. 4A depicts GLA and S1S3 PTase gene copy level in liver.

FIG. 4B depicts GLA and S1S3 PTase gene copy level in kidney.

FIG. 4C depicts GLA and S1S3 PTase mRNA levels in liver.

FIG. 4D depicts GLA and S1S3 PTase mRNA levels in kidney.

FIG. 5A is a graphical depiction of the two-promoter plasmid design for expression of S1S3 PTase and PPT1.

FIG. 5B depicts a SDS-PAGE and Coomassie stain of lysates of Expi293 cells transfected with the plasmid designs depicted in FIG. 5A.

FIG. 5C depicts a Western blot showing expression of S1S3 PTase in Expi293 cells transfected with the plasmid designs depicted in FIG. 5A.

FIG. 5D depicts PPT1 CI-MPR binding of conditioned media of the Expi293 cells transfected with the plasmid designs depicted in FIG. 5A.

FIG. 6A is a graphical depiction of the two-promoter AAV vector design for expression of S1S3 PTase and GBA1.

FIG. 6B depicts GCase activity of the Expi293 cells transfected with the plasmid designs depicted in FIG. 6A.

FIG. 6C depicts SDS-PAGE and Western blot showing expression of GCase and GAPDH in the Expi293 ells transfected with the plasmid designs depicted in FIG. 6A.

FIG. 6D depicts GCase CI-MPR binding of conditioned media of the Expi293 cells transfected with the plasmid designs depicted in FIG. 6A.

FIG. 7 depicts GCase activity in the liver, heart, and brain of mice injected with AAV9 viral vectors generated from the plasmid designs depicted in FIG. 6A.

FIG. 8A depicts GBA1 and S1S3 PTase gene copy number in liver, heart, brain, bone marrow, and spleen of mice injected with AAV9 viral vectors generated from the plasmid designs depicted in FIG. 6A.

FIG. 8B depicts GBA1 and S1S3 PTase mRNA copy number in liver, heart, brain, bone marrow, and spleen of mice injected with AAV9 viral vectors generated from the plasmid designs depicted in FIG. 6A.

FIG. 9 depicts GBA1 mRNA expression in the striatum of mice injected with AAV9 viral vectors generated from the plasmid designs depicted in FIG. 6A.

FIG. 10 depicts GCase protein expression in the somatosensory cortex of mice injected with AAV9 viral vectors generated from the plasmid designs depicted in FIG. 6A.

FIG. 11A is a graphical depiction of the two-promoter AAV vector design for expression of S1S3 PTase and HexM.

FIG. 11B depicts the HexM activity in conditioned media of Expi293 cells transfected with the plasmid design depicted in FIG. 11A.

FIG. 11C depicts the HexM activity in the cell lysate of Expi293 cells transfected with the plasmid design depicted in FIG. 11A.

FIG. 11D depicts the PTase activity in the cell lysate of Expi293 cells transfected with the plasmid design depicted in FIG. 11A.

FIG. 11E depicts the HexM CI-MPR binding of conditioned media of Expi293 cells transfected with the plasmid design depicted in FIG. 11A.

### DETAILED DESCRIPTION

The current invention is directed to novel compositions comprising expression vectors and gene therapy vectors and methods for generating lysosomal enzymes operably linked to a S1S3 variant of GlcNAc-1-Phosphotransferase (S1S3 PTase). The disclosure provides a novel two-promoter vector in which a lysosomal enzyme is under the control of one promoter and S1S3 PTase is under the control of a second promoter. Upon co-expression, the two-promoter vector produces lysosomal enzymes with appropriately phosphorylated N-linked oligosaccharides.

The expression of S1S3 PTase and lysosomal enzyme significantly increases the M6P content of the lysosomal enzyme being expressed. Having well phosphorylated enzymes allows for the efficient uptake and lysosomal delivery of the enzyme. This enables for better tissue distribution, cellular uptake, lysosomal targeting and substrate reduction. The two-promoter vector allows the expression of lysosomal enzyme and S1S3 PTase is regulated separately in gene transcription and translation which could increase the lysosomal enzyme expression level in cell, avoids complications caused by using longer bicistronic messages, and allows for expression of only the coding sequences (avoids the inclusion of additional amino acid sequences). The expression of the S1S3 PTase significantly increases the M6P content level in lysosomal enzymes.

### Definitions

Unless defined otherwise, all technical and scientific terms used herein have the same meaning as commonly understood by one of ordinary skill in the art to which the invention pertains. In describing and claiming the present invention, the following terminology will be used.

The term “expression vector” as used herein refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments, the disclosed vector is referred herein as a viral vector. In some embodiments, the disclosed vector is referred herein as an expression vector.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, com oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the vims particle or infectious units generated from vector of the invention which is required to prevent the particular disease condition, or which reduces the severity of and/or ameliorates the disease condition or at least one symptom thereof or condition associated therewith.

### Compositions of the Disclosure

The disclosure provides a composition comprising a vector comprising a polynucleotide encoding a lysosomal enzyme controlled by a first promoter and a polynucleotide encoding a modified GlcNAc-1 phosphotransferase controlled by a second promoter.

In some embodiments, the two-promoter vector comprises a particular nucleic acid sequence. In other embodiments, the two-promoter vector comprises a nucleic acid sequence having at least 20%, at least 30%, at least 40%, at least 50%, at least 60%, at least 70%, at least 75%, at least 80%, at least 85%, at least 90%, at least 95%, at least 99% similarity with SEQ ID NO: 1.

In some embodiments, the polynucleotide encoding a modified GlcNAc-1 PTase comprises a nucleic acid sequence corresponding to SEQ ID NO: 4 as disclosed in WO2021003442, the contents of which are incorporated fully herein. In other embodiments, the GlcNAc-1 PTase is encoded by a polynucleotide having at least 50%, at least 60%, at least 70%, at least 75%, at least 80%, at least 85%, at least 90%, at least 95%, at least 99% similarity with of SEQ ID NO: 4 as disclosed in WO2021003442.

In some embodiments, the polynucleotide encoding a lysosomal enzyme encodes a lysosomal enzyme involved in at least one LSD. Such lysosomal enzymes are known in the art and include, by way of example but not limitation, those listed in Table 1A-1C of WO2021003442, the contents of which are incorporated fully herein. By way of example but not limitation, in some embodiments, the lysosomal enzyme is selected from the group consisting of b-glucocebrosidase (GCase, GBA), Galactosylceremidase (GALC), a-Galactosidase (GLA), a-N-acetylglucosaminidase (NAGLU), acid a-glucosidase (GAA), b-hexosaminidase (HexM) and lysosomal acid a-mannosidase (LAMAN). In some embodiments, the polynucleotide encoding the lysosomal enzyme comprises a nucleic acid sequence of SEQ ID NOs: 5-10 of WO2021003442, the contents of which are incorporated fully herein.

In some embodiments, the two-promoter vector comprises an outside-in configuration, wherein the two promoters are in a distal configuration with the 3′ UTR regions for each gene adjacent.

In some embodiments, the two-promoter vector comprises an inside-out configuration, wherein the 5′ ends of both promoters are adjacent to drive gene expression outwards.

### Promoters

The promoters of the two-promoter vector may be constitutive, inducible/repressible, and/or cell type specific. In certain embodiments, the promoter may be constitutive. Non-limiting examples of constitutive promoters for mammalian cells include Cytomegalovirus (CMV), UBC, EF1alpha (EFS), JeT, SV40, PGK, CAG, CBA/CAGGS/ACTB, CBh, MeCP2, U6, and HI, including wild-type and known modifications thereof. Non-limiting examples of inducible promoters for mammalian cells include tetracycline, heat shock, steroid hormone, heavy metal, phorbol ester, adenovirus El A element, interferon, and serum inducible promoters. Without excluding other cell type specific promoters known in the art, some non-limiting examples of cell type specific promoters for neurons (e.g. syapsin), astrocytes (e.g. GFAP), oligodendrocytes (e.g. myelin basic protein), microglia (e.g. CX3CR1), neuroendocrine cells (e.g. chromogranin A), muscle cells (e.g. desmin, Mb), or cardiomyocytes (e.g. alpha myosin heavy-chain promoter) could be used. In an embodiment, a cell type specific promoter may be the Nrl (rod photoreceptor-specific) promoter or the HBB (haemoglobin beta) promoter.

The promoters of the two-promoter vector may both be constitutive, inducible/repressible, and/or cell type specific. Alternatively, the promoters of the two-promoter may be different types of promoters. By way of example but not limitation, the first promoter may comprise a constitutive promoter and the second promoter may comprise an inducible/repressible promoter. Alternatively, the first promoter may comprise a constitutive promoter and the second promoter may comprise a cell type specific promoter. Alternatively, the first promoter may comprise an inducible/repressible promoter and the second promoter may comprise a constitutive promoter. Alternatively, the first promoter may comprise an inducible/repressible promoter and the second promoter may comprise a cell type specific promoter. Alternatively, the first promoter may comprise a cell type specific promoter and the second promoter may comprise a constitutive promoter. Alternatively, the first promoter may comprise a cell type specific promoter and the second promoter may comprise an inducible/repressible promoter.

By way of example but not limitation, the promoter of the SIS3 PTase gene may be EFS or JeT and the promoter of the lysosomal enzyme gene may be CBH.

A vector of the present disclosure may further comprise one or more specific 3′ untranslated regions (3′ UTR, also named as Poly A sequence). In some embodiments, the 3′ UTR may be bidirectional. Non-limiting examples of 3′ UTRs include CMV, SP1, SV40 poly-A, bovine growth hormone poly-A (bGH polyA), rabbit-beta globin poly-A, and bidirectional UTRs such as sequences from SV40.

The 3′ UTR of the two-promoter vector may both be the same. Alternatively, the 3′ UTR of the two-promoter may be different types of 3′ UTR. By way of example but not limitation, the SIS3 PTase gene may be enhanced by SV40 and the lysosomal enzyme gene may be enhanced by bGH poly-A.

### Reporter/Selectable Marker Genes

In order to assess the expression of the polypeptides within the two-promoter vector, the vector can also comprise either a selectable marker gene or a reporter gene or both to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include those known in the art, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Non-limiting examples of reporter genes include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Introducing Genes Into A Cell

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments in which a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al, 1991 Glycobiology 5:505-10). However, compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, without limitation, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### Vectors

The vectors to be used for treating or preventing LSDs in a subject as disclosed herein, are suitable for replication and, optionally, integration in eukaryotic cells.

Typical vectors contain transcription and translation terminators, initiation sequences, and promoters useful for regulation of the expression of the desired nucleic acid sequence.

The vectors of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The isolated nucleic acid of the disclosure can be cloned into a number of types of vectors. For example, the nucleic acid can be cloned into a vector including, but not limited to a plasmid, a phagemid, a phage derivative, an animal virus, and a cosmid. Vectors of interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals.

Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In a preferred embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce post-mitotic cells in a stable and efficient manner. Expression of a particular gene contained within an AAV vector can be specifically targeted to one or more types of cells by choosing the appropriate combination of AAV serotype, promoter, and delivery method

In some embodiments, the disclosed two-promoter viral vector comprises an adenovirus (e.g. Ad-SYE, AdSur-SYE, Ad5/3-MDA7/IL-24, Ad-SB, Ad-CRISPR, oncolytic Ad); an adeno-associated virus, AAV (e.g. AAV-MeCP2, AAV1, AAV5, Dual AAV9 AAV8, AAV9, AAVrh1O, AAVhu37); a herpes simplex virus, HSV (e.g. HSV1, HSV2, HSV-1, HF10 Oncolytic HSV-2); a Retrovirus (e.g. RRV/Toca 511, GRV); a lentivirus (e.g. HIV-1, HIV-2); an alphavirus (SFV, MI); a flavivirus (Kunjin virus); a rhabdovirus (VSV); a measles virus (e.g. MV-Edm); a Newcastle disease virus (e.g. NDV90); an anhinga Picomaviruses Coxsackievirus (e.g. CVB3, CAV21, EV1); or a poxvirus (e.g. PANVAC, VV, VV-GLV-1h153, CPXV).

In some embodiments the disclosed two-promoter vector is a viral vector comprising an adenovirus, an adeno-associated viruses (AAV), an alphavirus, a flavivirus, a herpes simplex virus (HSV), a measles virus, a rhabdovirus, a retrovirus, a lentivirus, a Newcastle disease virus (NDV), a poxvirus, or a picomavirus. In some embodiments the two-promoter vector is a viral vector comprising an adenovirus, an adeno-associated viruses (AAV), a retrovirus, or a lentivirus.

In some embodiments, the polynucleotide encoding a lysosomal enzyme and a polynucleotide encoding a modified GlcNAc-1 PTase are contained within an AAV vector. More than 30 naturally occurring serotypes of AAV are available. Many natural variants in the AAV capsid exist, allowing identification and use of an AAV with properties specifically suited for skeletal muscle. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, to name a few.

The use of AAVs is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector; it has been widely used for efficient gene transfer experiments in different target tissues and animal models. Clinical trials of the experimental application of AAV2 based vectors to some human disease models are in progress, and include therapies for diseases such as for example, cystic fibrosis and hemophilia B. Other useful AAV serotypes include AAVI, AAV3, AAV4, AAV5, AAV6, AAV7, AAV8, and AAV9.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vpl capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of a lysosomal enzyme of interest and a modified GlcNAc-1 PTase, include AAV2/8 (see U.S. Pat. No. 7,282,199), AAV2/5 (available from the National Institutes of Health), AAV2/9 (International Patent Publication No. W02005/033321), AAV2/6 (U.S. Pat. No. 6,156,303), and AAVrh8 (International Patent Publication No. W02003/042397), among others.

In some embodiments, the two-promoter vector described herein contains sequences encoding a selected AAV serotype capsid, e.g., an AAV8 capsid, or a fragment thereof. In another embodiment, the two-promoter vector described herein contains sequences encoding a selected AAV serotype rep protein, e.g., AAV8 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV8 origin. Alternatively, the two-promoter vector described herein may contain rep sequences are from an AAV serotype differing from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282, 199.

A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a polynucleotide encoding a lysosomal enzyme and a polynucleotide encoding a modified GlcNAc-1 PTase; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Most suitably, such a stable host cell will contain the required component(s) under the control of a constitutive promoter. However, the required component(s) may be under the control of an inducible promoter. Examples of suitable inducible and constitutive promoters are provided elsewhere herein, and are well known in the art. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the disclosure may be delivered to the packaging host cell in the form of any genetic element which transfers the sequences carried thereon. The selected genetic element may be delivered using any suitable method, including those described herein and any others available in the art. The methods used to construct any embodiment of this disclosure are known to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N. Y). Similarly, methods of generating rAAV virions are well known and the selection of a suitable method is not a limitation on the present disclosure (see, e.g., K. Fisher et al, 1993 J. Virol., 70:520-532 and U.S. Pat. No. 5,478,745, among others).

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9 or other known or as yet unknown AAV serotypes. These ITRs or other AAV components may be readily isolated from an AAV serotype using techniques available to those of skill in the art. Such an AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

### Modified Cell

In some embodiments, the disclosure provides a cell comprising a vector of the disclosure.

The cell may be a prokaryotic cell or a eukaryotic cell. Appropriate cells include, but are not limited to, mammalian, bacterial, yeast, fungal, and insect cells.

In some embodiments, the disclosure provides a mammalian cell comprising a two-promoter vector of the disclosure. In some embodiments, the cell is a mammalian cell capable of expressing a human sequence and/or producing a human protein. In some embodiments, the mammalian cell is isolated or derived from a mouse, rat, guinea pig, rabbit, cat, dog, or non-human primate. In some embodiments, the cell is a human cell capable of expressing a human sequence and/or producing a human protein. In some embodiments, the cell is a cultured cell. In some embodiments, the cell is an immortalized or stabilized cell line. Non-limiting examples of mammalian cells that may be used for protein expression include Chinese hamster ovary (CHO) cells, HeLa cells, Human embryonic kidney 293 (HEK293) cells, baby hamster kidney (BHK) cells, monkey kidney cells (COS), human hepatocellular carcinoma cells (e.g. Hep G2), human embryonic kidney cells, Bos primigenius, and Mus musculus. In a specific embodiment, the cells are CHO cells. Additionally, the mammalian cell may be an established, commercially-available cell line (e.g., American Type Culture Collection (ATCC), Manassas, VA). The cell may be an immortalized cell. Alternatively, the cell may be a primary cell.

In some embodiments, the cell is a bacterial cell. Non-limiting examples of suitable bacterial cells include E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp. and alpha-proteobaeteria such as Wolbachia sp., cyanobacteria, spirochaetes, green sulfur and green non-sulfur bacteria, Gram-negative cocci, Gram negative bacilli which are fastidious, Enterobacteriaceae-glucose-fermenting gram-negative bacilli, Gram negative bacilli-non-glucose fermenters, Gram negative bacilli-glucose fermenting, oxidase positive. Particularly useful bacterial cells for protein expression include Gram negative bacteria, such as Escherichia coli, Pseudomonas fluorescens, Pseudomonas haloplanctis, Pseudomonas putida AC 10, Pseudomonas pseudoflava, Bartonella henselae, Pseudomonas syringae, Caulobacter crescentus, Zymomonas mobilis, Rhizobium meliloti, Myxococcus xanthus and Gram positive bacteria such as Bacillus subtilis, Corynebacterium, Streptococcus cremoris, Streptococcus lividans, and Streptomyces lividans. E. coli is one of the most widely used expression cells. Accordingly, the techniques for overexpression in E. coli are well developed and readily available to one of skill in the art. Further, Pseudomonas fluorescens, is commonly used for high level production of recombinant proteins (i.e. for the development bio-therapeutics and vaccines).

In some embodiments, the cell is a yeast or fungal cell. Particularly useful fungal cells for protein expression include Aspergillis oryzae, Aspergillis niger, Trichoderma reesei, Aspergillus nidulans, Fusarium graminearum. Particularly useful yeast cells for protein expression include Candida albicans, Candida maltose, Hansenula polymorpha, Kluyveromyces fragilis, Kluyveromyces lactis, Pichia guillerimondii, Pichia pastoris, Saccharomyces cerevisiae, Schizosaccharomyces pombe, and Yarrowia lipolytica.

In some embodiments, the cell is an insect cell. Non-limiting examples include Spodoptera frugiperda cell lines (such as the Sf9 or Sf21), drosophila cell lines, or mosquito cell lines (such as Aedes albopictus derived cell lines)

In some embodiments, the cell is a primary cell, modified to express a vector of the disclosure and cultured ex vivo. In some embodiments, the cultured cell is immortalized or otherwise modified to facilitate propagation of the cell in vitro indefinitely, generating a cultured cell line.

A cell comprising the disclosed two-promoter vector may be used for protein expression and, optionally, purification. Methods for expressing and, optionally, purifying an expressed protein from a cell are standard in the art.

In some embodiments, the cell comprising a two-promoter vector of the disclosure may be used to produce a polypeptide encoded by an enzyme construct of the disclosure. Generally, production of a polypeptide of the disclosure involves transfecting cells with a vector comprising an enzyme construct and then culturing the cells so that they transcribe and translate the desired polypeptide. The isolated cells may then be lysed to extract the expressed polypeptide for subsequent purification.

### Pharmaceutical Compositions and Formulations

Also provided herein is a pharmaceutical composition comprising a lysosomal enzyme expressed by the two-promoter vector of the disclosure.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-dose unit. In some embodiments, the presently disclosed compositions can be formulated in a natural capsid, a modified capsid, as a naked RNA, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In some embodiments, the disclosure provides a pharmaceutical composition for treating a subject suffering from LSDs. In some embodiments, the disclosure provides a pharmaceutical composition comprising a lysosomal enzyme expressed by a two-promoter vector of the disclosure and a pharmaceutically acceptable carrier.

Pharmaceutically acceptable carriers, which are useful, include, but are not limited to, glycerol, water, saline, ethanol and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The disclosed composition may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. In some embodiments, the preservative is a combination of about 0.5% to 2.0% benzyl alcohol and 0.05% to 0.5% sorbic acid.

The composition may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. In some embodiments, BHT and disodium edetate are the antioxidant and the chelating agent respectively for some compounds, however, other suitable and equivalent antioxidants and chelating agents may be substituted therefore as would be known to those skilled in the art.

The disclosure provides a pharmaceutical composition comprising a lysosomal enzyme expressed by a vector of the disclosure and a pharmaceutically acceptable carrier.

The compositions of the disclosure may be used for enzyme replacement therapy (ERT). Alternatively or in addition, the compositions of the disclosure may be used for gene therapy.

### Methods of the Disclosure

The disclosure provides method to treat a deficient lysosomal enzyme in a subject diagnosed with LSD or in a subject at risk for developing an LSD. The method improves phosphorylation of lysosomal enzymes thereby treating the subject or preventing the occurrence of the LSD in the subject. Further, the method improves quality of life in a patient.

The disclosure provides a method of treating a LSD, the method comprising administering to a subject a composition of the disclosure, thereby treating the LSD.

The disclosure provides a method of treating a LSD, the method comprising administering to a subject a therapeutically-effective amount of a composition of the disclosure, wherein the composition increases phosphorylation of a lysosomal enzyme, thereby treating the LSD.

The disclosure provides a method of treating a subject suffering from a LSD, the method comprising administering to the subject a pharmaceutical composition of the disclosure, thereby increasing the phosphorylation of a lysosomal enzyme and treating the subject.

The disclosure provides a method of preventing the occurrence of a LSD in a subject in need thereof, the method comprising administering to the subject a pharmaceutical composition of the disclosure, thereby increasing the phosphorylation of a lysosomal enzyme and preventing the occurrence of a LSD in the subject.

The disclosure provides a method of ameliorating the phosphorylation of a lysosomal enzyme responsible for a LSD in a subject in need thereof, the method comprising administering to the subject a composition of the disclosure, wherein the composition increases the phosphorylation of the lysosomal enzyme.

### Administration/Dosing

The regimen of administration may affect what constitutes an effective amount. For example, the therapeutic formulations may be administered to the patient subject either prior to or after a surgical intervention related to a LSD, or shortly after the patient was diagnosed with a LSD. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient subject, preferably a mammal, more preferably a human, may be carried out using known procedures, at dosages and for periods of time effective to treat a LSD in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non limiting example of an effective dose range for a therapeutic compound of the disclosure is from about 0.01 and 50 mg/kg of body weight/per day.

The compound can be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of LSDs.

### Routes of Administration

One skilled in the art will recognize that although more than one route can be used for administration, a particular route can provide a more immediate and more effective reaction than another route.

Routes of administration of the disclosed compositions include inhalational, oral, nasal, rectal, parenteral, sublingual, transdermal, transmucosal (e.g., sublingual, lingual, (trans) buccal, (trans) urethral, vaginal (e.g., trans-and perivaginally), (intra) nasal, and (trans) rectal), intravesical, intrapulmonary, intraduodenal, intragastrical, intrathecal, intra-cisterna magna (ICM), intraspinal, intraventricular, intracerebroventricular, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein. In one embodiment, the treatment of LSD comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal and any combination thereof.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein the vector is complexed to another entity, such as a liposome, aggregated protein or transporter molecule.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present disclosure (for instance, the cost associated with synthesis). One skilled in the art can easily make any necessary adjustments in accordance with the exigencies of the particular situation.

Cells containing the therapeutic agent may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk:: Tmk) and AZT; and deoxycytidine kinase and cytosine arabinoside.

### EXAMPLES

The disclosure is described with reference to the following Examples. These Examples are provided for the purpose of illustration only and the disclosure should in no way be construed as being limited to these Examples, but rather should be construed to encompass any and all variations which become evident as a result of the disclosure provided herein.

Without further description, it is believed that one of ordinary skill in the art can, using the preceding description and the following illustrative examples, make and utilize the compounds of the present disclosure and practice the claimed methods. The following working examples are not to be construed as limiting in any way the present disclosure.

### Example 1: Construction of SIS3-GLA Two-Promoter Vector

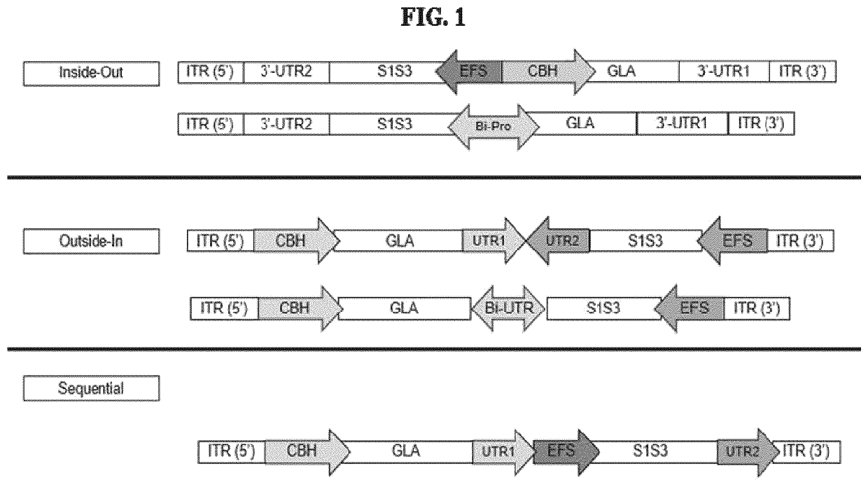

To generate vectors containing two promoters, DNA fragments were assembled to generate gene expression cassettes where SIS3 PTase and lysosomal enzyme GLA can be expressed from separate promoters and separate 3′ UTRs. Fragments were assembled in various compositions such that the lysosomal enzyme is driven by a strong promoter (i.e. Cbh promoter) with a standard 3′ UTR (i.e. bovine Growth Hormone polyA), and S1S3 PTase is driven by a separate promoter (i.e. EF-1-alpha core—Efs or modified JeT promoter) with a separate 3′ UTR (i.e. sv40 poly A). These two gene expression cassettes were then assembled in plasmids for generation of AAV such that (1) the 5′ ends of both promoters are adjacent—Inside-out, (2) the 3′ UTR regions are adjacent—Outside-In, or (3) the two cassettes are arranged sequentially (3′ UTR of cassette 1 adjacent to the 5′ region of the second promoter)—Sequential (see FIG. 1).

The DNA fragments containing the promoters (such as EFs or JeT) and UTR (Such as: sv40, bGH polyA) were ordered from IDT or Twist Biosciences (https://www.idtdna.com/pages/products/genes-and-gene-fragments/double-stranded-dna-fragments/gblocks-gene-fragments), Twist Bioscience (https://www.twistbioscience.com/) We lead innovation in DNA synthesis. These fragments were assembled using restriction enzymes and standard molecular biology techniques that allowed for the creation of cassettes containing a lysosomal enzyme or SIS3 PTase. Restriction sites (i.e. SacII, BamHI, Nhel) were placed to allow the replacement of lysosomal enzymes or modify the SIS3 PTase sequence (Xbal/PmeI). DNA fragments were assembled to create the final plasmid that contains ITR sequences from AAV, using standard molecular biology techniques (restriction digest, ligation, bacterial transformation).

Gene expression cassettes were arranged in three orientations (FIG. 1). The first, is described as Inside-Out with the promoters adjacent to drive gene expression outwards with the 3′-UTR sequences on opposite ends. The promoter used could be two different promoters such as Cbh, EFs, JeT or a bi-directional promoter. The second orientation, Outside-In, features the promoters in a distal configuration with the 3′-UTR region of the cassettes adjacent. This allows for genes to be driven by distinct promoters, such as Cbh, EFs, JeT, and the 3′-UTR can be bovine growth hormone poly A, sv40 poly A, rabbit-beta globin poly A, or bidirectional UTRs such as a sequence from sv40. The third orientation is a sequential orientation where the gene expression cassettes are placed consecutively on the same strand. Sequential orientation has a 5′ promoter with the first gene and then a 3′ UTR, followed by a second promoter, gene and second UTR consecutively. These different orientations allow for the expression of mRNA from individual promoters that can be used to generate proteins for SIS3 PTase with a lysosomal enzyme. This configuration allows for a specific promoter and UTR set to provide controlled gene expression of each gene individually by using separate gene cassettes. FIG. 1 shows how GLA and S1S3 PTase can be expressed using two promoters. FIG. 1 shows configurations in an AAV expression cassette. These two-promoter arrangements for co-expression of S1S3 PTase can be used for protein expression in enzyme-replacement therapies, AAV based gene therapies, nano-particle based technologies.

### Example 2: Transfection of Inside-Out Designs of the Two-Promoter Vector into HEK293T Cells

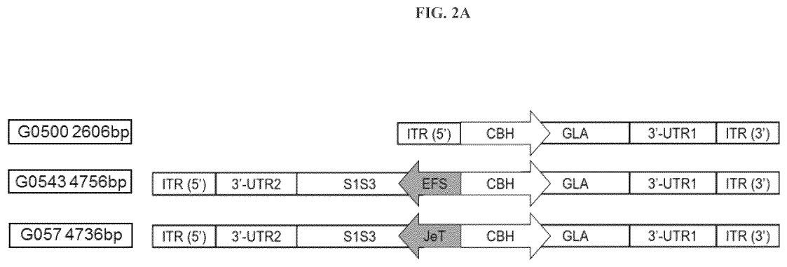

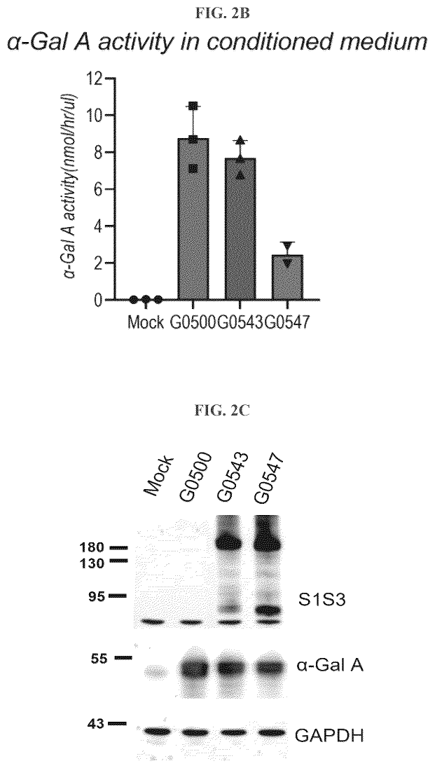

Initially testing of the two-promoter Inside-Out designs for co-expression of S1S3 PTase with a lysosomal enzyme (as disclosed in Example 1) was performed using transient transfection of HEK293T cells. First cells were transfected with the inside-out plasmids ((G0543—Efs-S1S3-sv40/Cbh-GLA-bGH poly A), (G0547—JeT-S1S3-sv40/Cbh-GLA-bGH poly A)) or a control (G0500—Cbh-GLA-sv40) (FIG. 2A). Cell lysates were assayed for GLA activity. Plasmid transfection increased GLA activity was detectable for the control (G0500) and for the inside-out two promoter system for GLA/S1S3 PTase expression (G0543 or G0547) compared to the mock transfected control (FIG. 2B). Expression of GLA protein and S1S3 PTase protein was confirmed by Western blot (FIG. 2C). These panels demonstrate the function of the inside-out two-promoter configuration.

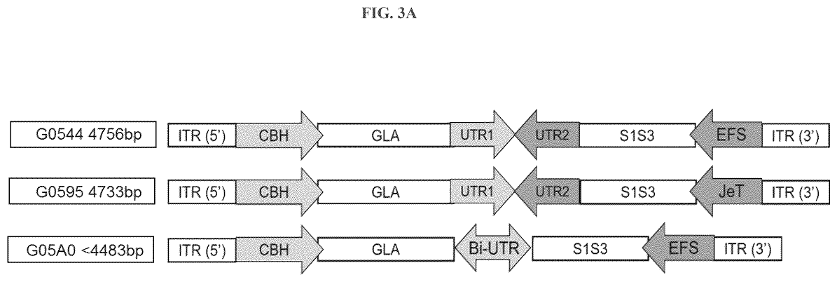

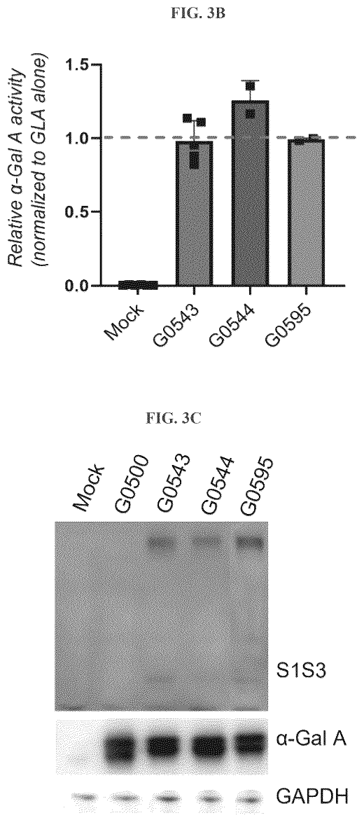

Next the two-promoter Outside-In designs for co-expression of S1S3 PTase with a lysosomal enzyme was performed using transient transfection of HEK293T cells. First cells were transfected with the outside-in plasmids G0544 (Efs-S1S3-sv40/Cbh-GLA-bGH poly A), G0595 (JeT-S1S3-sv40/Cbh-GLA-bGH poly A), the inside-out construct G0543 (Efs-S1S3-sv40/Cbh-GLA-bGH poly A), or a control (G0500-Cbh-GLA-sv40) (FIG. 3A). Cell lysates were assayed for GLA activity. Again, plasmid transfection increased GLA activity was detectable for the control (G0500), the inside-out two promoter system (G0543) and using the outside-in two-promoter system for GLA/S1S3 expression (G0544 or G0595) compared to the mock transfected control (FIG. 3B). Expression of GLA protein and S1S3 PTase protein was confirmed by Western blot (FIG. 3C). These panels demonstrate the function of the inside-out two-promoter configuration.

### Methods.

HEK-293T cells were transfected using lipofectamine 3000 with plasmids for AAV cassettes containing two-promoters, one (e.g. Cbh) drives GLA expression, and the other (e.g. EFs) drives S1S3 PTase expression. 48 h after transfection cells were harvested, conditioned media and cell lysates were saved for analysis by enzyme activity assays, CIMPR binding, and Western blot.

α-GAL activity was measured following an established protocol—see Mayes, Scheerer et al. 1981, Yasuda, Huston et al. 2020.

Briefly, assays were performed in 0.1M-0.2M Citrate-Phosphate buffer, pH4.5, 0.25% TX-100. α-GAL substrate was 4-methylumbelliferyl α-D-galactopyranoside, Cat: M7633, Millipore-Sigma) dissolved in DMSO diluted to 1-5 mM in assay buffer. Assay buffer includes a Gal-B inhibitor (GalNAc) N-acetyl-Galactosamine, Cat: A2795, Millipore-Sigma 90mM. 45 ul of substrate solution was aliquoted into a 96 well plate, and 5 ul of sample (cell lysate, conditioned media, purified protein, etc.) were added in duplicate (2 wells per sample). To compare activity, serial dilutions of 4-MU (4-Methylumbelliferone sodium salt, Sigma, M1381 in the same plate during the assay. The plate was then sealed and incubated at 37° C. for 1 hr with shaking/agitation. Reactions were stopped by addition of 150 ul of stop buffer (0.4 M glycine-NaOH, pH 10.8.) α-GAL activity was then measured using substrate fluorescence at Ex 360, Em 460 using a Molecular Devices Spectramax Id3 plate reader and Softmax Pro 7.1 software. Background fluorescence was subtracted based on substrate alone or mock transfection conditions. Analysis was performed in Excel and graphs generated using GraphPad Prism software. Protein concentrations for samples were measured using BCA protein assay (ThermoFisher 23227) following the manufacturer's protocol. Absorbance at 260 was then measured using a Molecular Devices Spectramax Id3 plate reader and Softmax Pro 7.1 software. Concentrations were compared to BSA standards.

### Example 3: Two-Promoter Vector Expression In Vivo

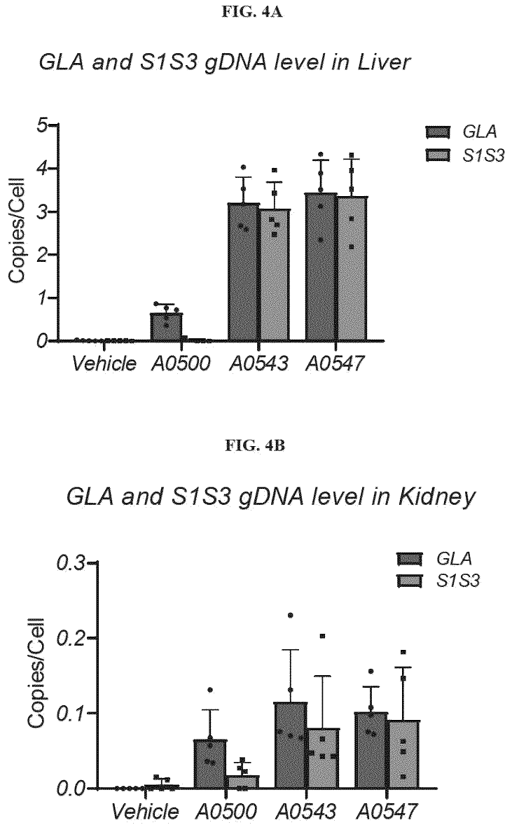

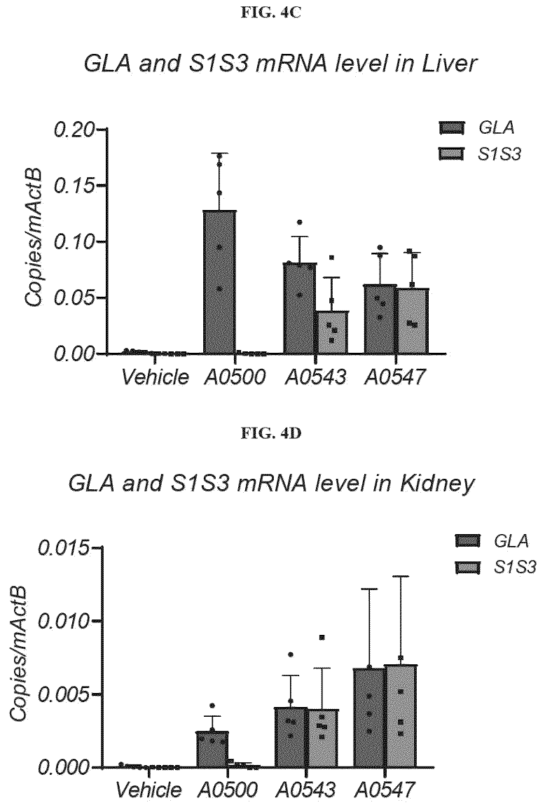

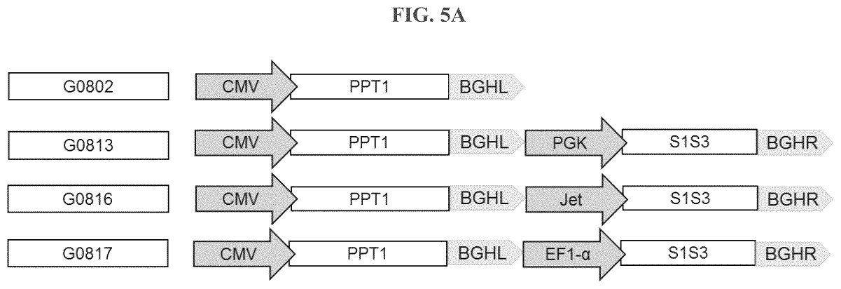

To determine the expression of AAV9 gene therapy with the two-promoter transgene of GLA and S1S3 PTase, 8 weeks old wild type C57BL/6J mice were dosed with a moderate dose of GLA alone AAV9 or GLA with S1S3 PTase in two-promoter inside-out configuration. 2 weeks later after AAV9 injection, tissues were recovered and homogenized. Gene copy and its mRNA level were measured by digital droplet PCR. Comparing with A0500—GLA alone AAV9, the two two-promoter AAV9 vectors show higher GLA and S1S3 PTase gene copy level in Liver and Kidney (FIG. 4A and FIG. 4B), and mRNA levels (FIG. 4C and FIG. 4D).

### Example 4: Sequential Promoter Designs for Co-Expressing PPT1 and S1S3 PTase

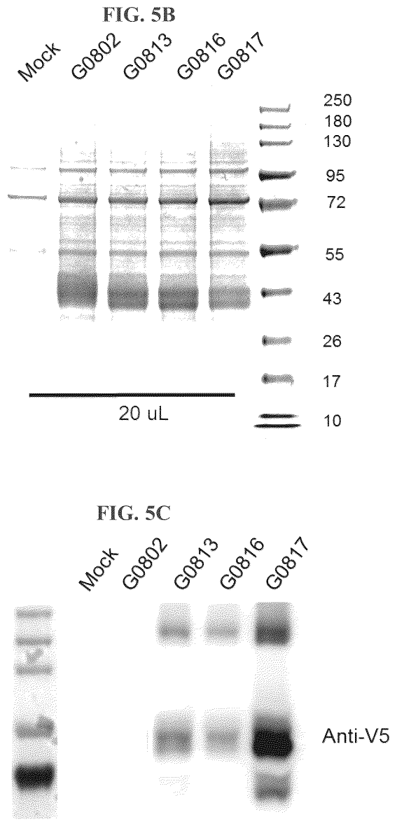

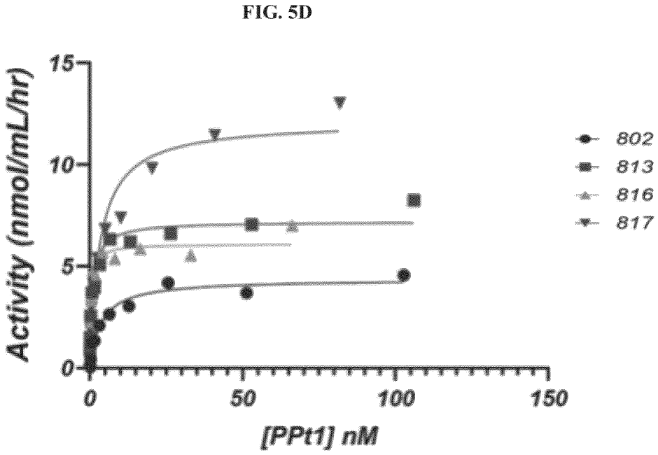

Sequential promoter designs were used for co-expression of PPT1 with S1S3-phosphotransferase for transfection in Expi293 suspension cells. (FIG. 5A) shows sequential plasmid designs with CMV promoter for expression of PPT1, and a second promoter for S1S3 expression (PGK, JeT, EF1-alpha), G0802 is a plasmid for expression of PPTI alone. Expi293 cells were transfected with the indicated plasmids, conditioned media was harvested 48 h after transfection, and expression of PPT1 examined by SDS-PAGE and Coomassie stain (FIG. 5B). All PPT1 containing plasmids demonstrate comparable PPT1 enzyme/protein expression. The effect of S1S3 expression was examined by western blotting to V5 tag fused to the c-terminal of S1S3 gene in cell lysate. As shown in FIG. 5C, all promoters for SIS3 yield good expression of SIS3, and EF1-α gives the best expression. Cation-independent mannose 6-phosphate receptor (CI-MPR) binding assay was also performed with the conditioned media from transfected cells. Binding was determined using a PPT1 activity assay. Plasmids containing sequential promoters for S1S3 expression demonstrate enhanced PPT1 binding to CI-MPR (G0813, G0816, G0817) compared to PPT1 expressed alone (G0802) (FIG. 5D).

### Example 5: Two-Promoter Designs for Expression of GBA1 via AAV Vectors

### In Vitro.

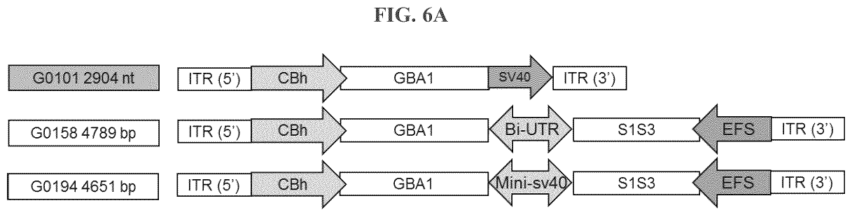

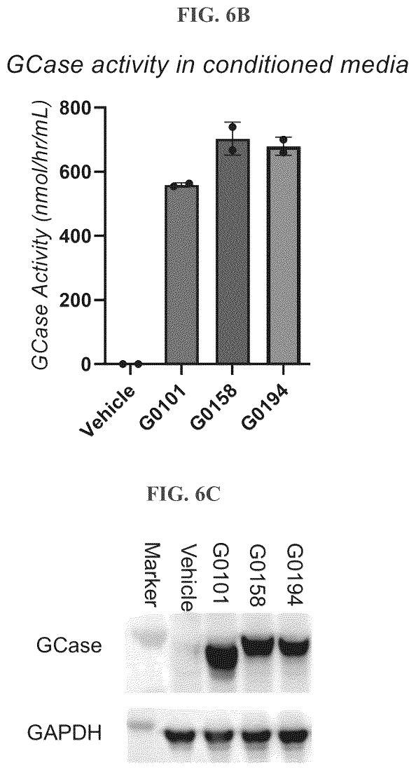

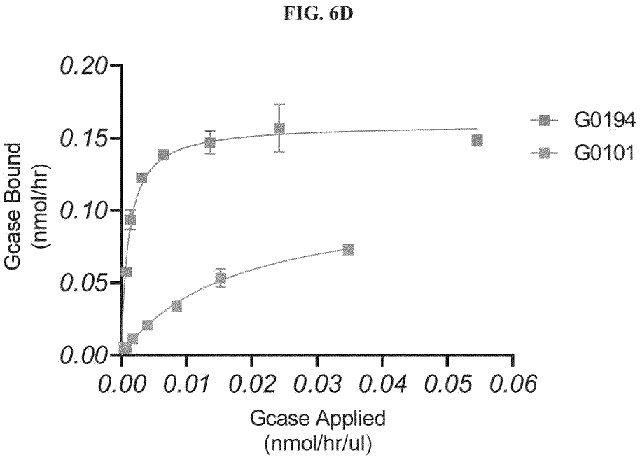

The effect of two-promoter design was assessed using AAV vectors. Plasmids were designed containing AAV ITR sequences and cassettes for expression of GBA1 gene using the Cbh promoter. In addition, an outside in design was used to co-express SIS3 using the short EF1-a promoter (EFs) (G0158 and G0194). Co-expression plasmids were compared to a plasmid expressing GBA1 alone (G0101) (FIG. 6A).

Outside-In AAV plasmids were transfected in Expi293 suspension cells. Cells were transfected with GBA1 alone (G0101) or GBA1/S1S3 co-expression plasmids (G0158 and G0194) and conditioned media was harvested 48 h post-transfection. Conditioned media was assayed for GCase enzyme activity using an in vitro activity assay. Minimal activity is detectable for vehicle transfected cells, while comparable GCase activity is present from cells transfected with (G0101, G0158 or G0194) (FIG. 6B). Conditioned media (from FIG. 6B) was examined by SDS-PAGE and Western blot, using anti-GCase (top) or anti-GAPDH loading control (bottom) (FIG. 6C). Similar amounts of GCase are present for G0101, G0158, or G0194, while minimal GCase is present in media from vehicle control. (NOTE size shift due to K360N mutation in G0158/G0194). Conditioned media (From FIG. 6B) was assessed for binding to the CI-MPR. CI-MPR was pre-bound to a 96 well dish and incubated with conditioned media from G0101 or G0194 expressing cells. Binding was determined using a GCase activity assay (FIG. 6D). G0194 (orange) demonstrates enhanced binding to the CI-MPR compared to G0101 (gray).

### In Vivo Animal Study.

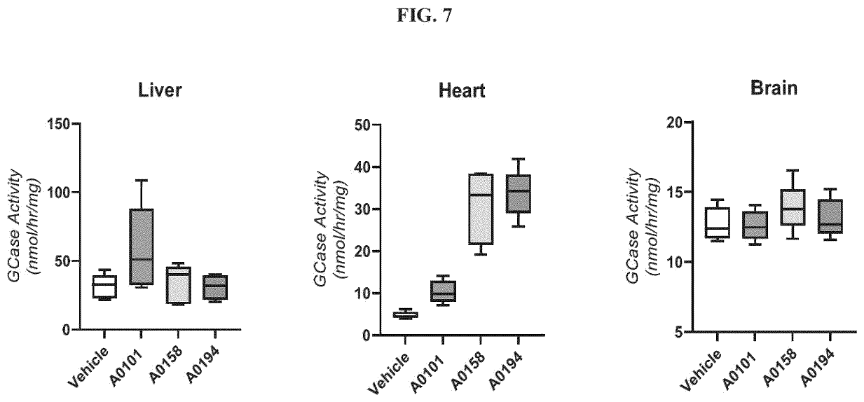

Plasmids as described in (FIG. 5A) were used to generate AAV9 viral vectors for expression of GBA1 alone (G0101) or co-expression with S1S3 (G0158 and G0194). Wild-type mice were injected with 2e13 vg/kg of AAV9 or formulation buffer as control intravenously (IV) through tail vein. 21 days post-injection, animal tissues were harvested, homogenized and GCase activity in lysate was measured. GCase activity was detected in liver, heart and brain (FIG. 7).

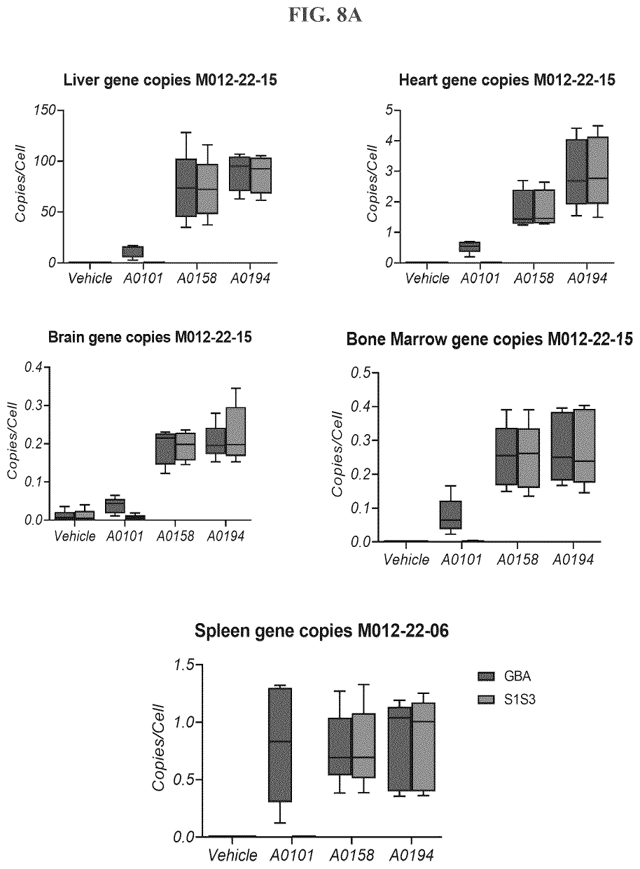

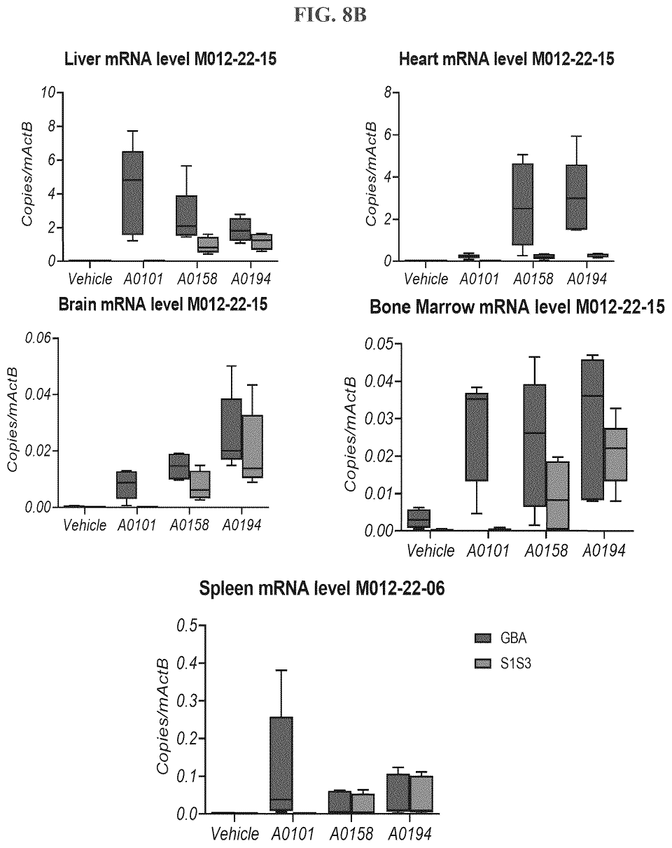

Tissues were also processed for analysis of GBA1 and S1S3 genomic DNA and mRNA from liver, heart, brain, bone marrow, and spleen. DNA and mRNA were analyzed by ddPCR using specific primers and probes to detect GBA1 and SIS3. As shown in FIGS. 8A-8B, increased DNA (FIG. 8A) and mRNA (FIG. 8B) copies of GBA1 were detected in all tissues for animals treated with AAV (G0101, G0158, G0194; Blue). Increased DNA (FIG. 8A) and mRNA (FIG. 8B) copies of S1S3 were detected in all tissues for animals treated with AAV (G0158, G0194; Orange). This demonstrates that the outside-in AAV9 can co-express both GBA1 and S1S3 (G0158 and G0194).

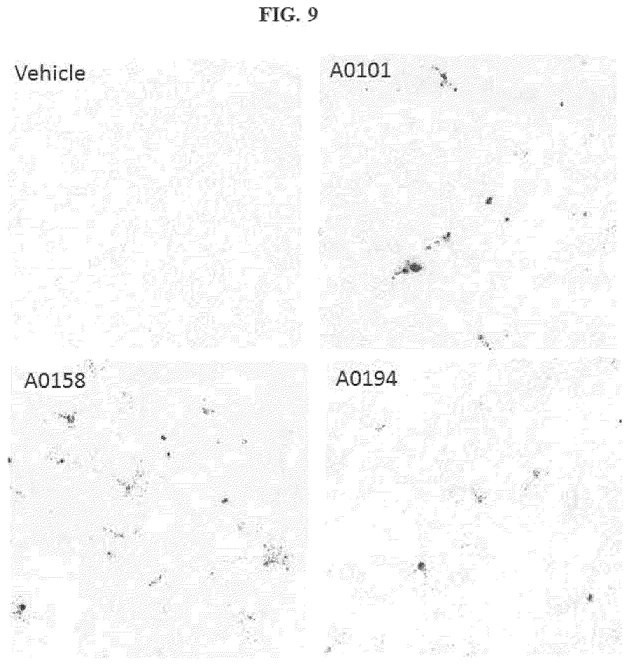

To better understand the AAV9 transduction in mouse brain through systemic injection (IV), mouse brain was examined by Basescope to assess GBA1 transcription in situ. The striatum was examined by microscopy to visualize GBA1 mRNA expression and distribution in the striatum from mouse brain. Panels demonstrate similar GBA1 mRNA expression for all mouse brains treated with AAV9 containing GBAl gene (FIG. 9). No GBA1 is detectable in vehicle treated controls.

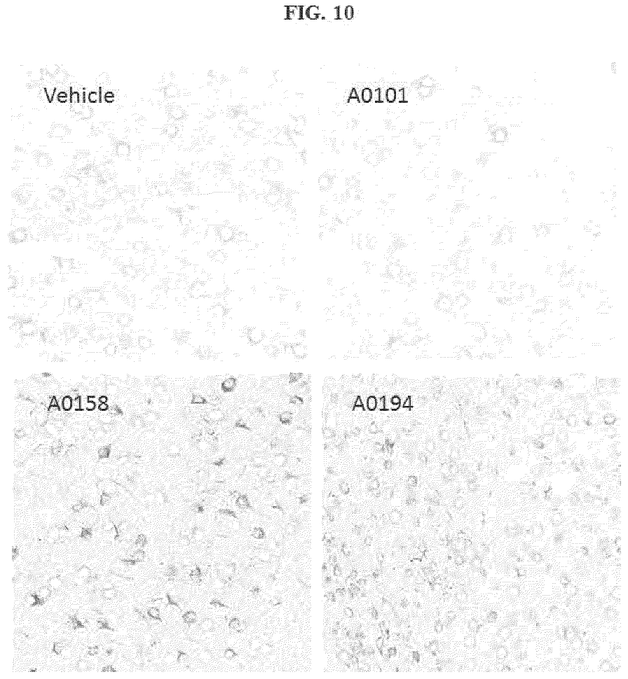

Mouse brain was also examined by immunohistochemistry staining using human GCase specific antibody to detect GCase enzyme/protein in somatosensory cortex of mouse brain. Panels demonstrate enhanced GCase staining for mice treated with GBA1-S1S3 co-expression AAV9 (G0158 and G0194) (FIG. 10). This suggests that there is enhanced tissue distribution of GCase when co-expressed with S1S3 using our dual-promoter AAV design.

### Example 6: Two-Promoter Designs for Expression of HexM via AAV Vectors

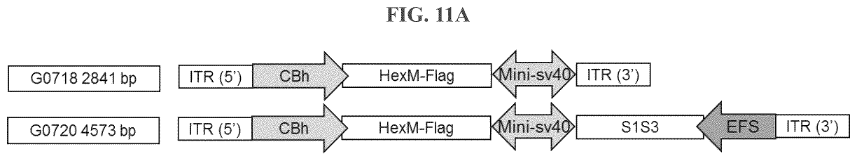

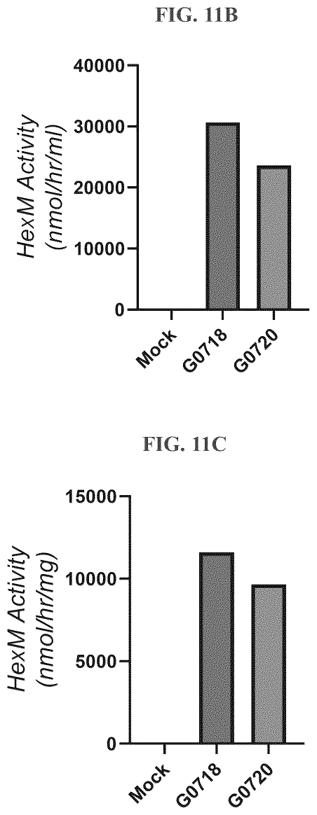

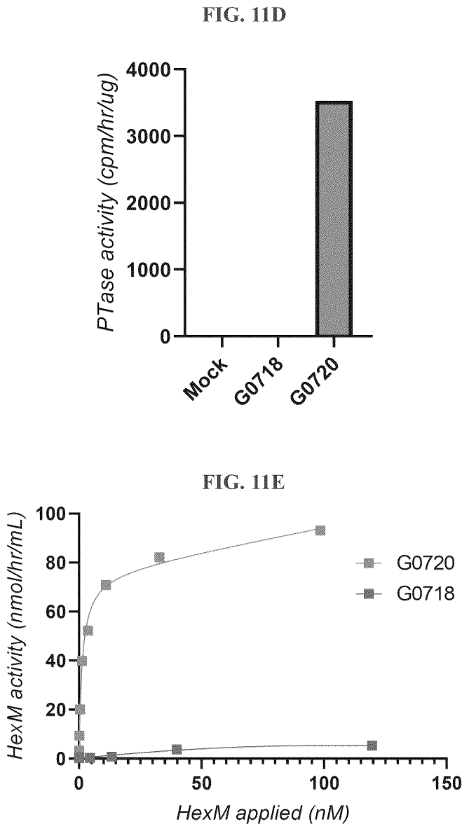

Plasmids were designed containing AAV ITR sequences and cassettes for expression of HexM (for description of HexM, see Tropak, MB, Yonekawa, S, Karumuthil-Melethil, S, Thompson, P, Wakarchuk, W, Gray, SJ, et al., Construction of a hybrid β-hexosaminidase subunit capable of forming stable homodimers that hydrolyze GM2 ganglioside in vivo. Mol. Ther.-Methods Clin. Dev. 3:15057 (2016)) with a C-tail FLAG tag (FIG. 11A), using the Cbh promoter. In addition, an outside in design was used to co-express SIS3 using the short EF1-a promoter (EFs) (G0720). Co-expression plasmids were compared to a plasmid expressing FLAG tagged HexM alone (G0718).

Outside-In AAV plasmids were transfected in Expi293 suspension cells. Cells were transfected with HexM alone (G0718) or HexM/S1S3 co-expression plasmids (G720) and conditioned media and cell lysates were harvested 48 h post-transfection. Conditioned media was assayed for HexM enzyme activity using 4-methylumbelliferyl-7-(6-sulfo-2-acetamido-2-deoxy-β-D-glucopyranoside) sodium salt (MUGS, sigma, M0662). Minimal activity is detectable for vehicle transfected cells, while comparable HexM activity is present from cells transfected with (G0718 or G0720) (FIG. 11B). Cell lysates from transfected Expi293 cells were assayed for HexM activity by 4-MU substrate MUGS (FIG. 11C) and phosphotransferase (PTase) activity (Lin Liu etc, Mol Ther Methods Clin Dev. 2017 Mar. 29;5:59-65) (FIG. 11D). Similar HexM activity is detected in cell lysates from cells transfected with HexM plasmids (G0718 or G0720). Only cells transfected with S1S3 expression plasmid (G0720) show enhanced PTase activity compared to vehicle or HexM alone expressing cells (FIG. 11D). Conditioned media (from FIG. 11B) was assessed for binding to the CI-MPR. CI-MPR was pre-bound to a 96 well dish and incubated with conditioned media from G0718 or G0720 expressing cells. Binding was determined using a HexM activity assay (FIG. 11E). G0720 (orange) demonstrates enhanced binding of HexM to the CI-MPR compared to G0718 (gray).

Methods: The wild type mice were purchased from the Jackson laboratory and transferred to Sanford Research center for animal studies. The mouse was maintained under a 12 hr light/12 hr dark cycle. Animals were administered intravenously (IV) through tail vain at 8 weeks old with 5 animals in each group. 3 weeks post-injection, animals were sacrificed and perfused with 10 mL cold PBS buffer before tissue harvesting. The consistent area from each tissue was collected either for snap-frozen or fixation at 4% formaldehyde. At Sanford research center, viability observations for pain, moribundity and mortality were performed, and body weight was assessed daily. No animals were noted any significant changes during the study.

(1-28.) canceled

29. A composition comprising a vector comprising a sequence encoding a first promoter operably linked to a first polynucleotide encoding a lysosomal enzyme and a second promoter operably linked to a second polynucleotide encoding a modified GlcNAc-1 phosphotransferase.

30. The composition of claim 1, wherein the vector is a viral vector.

31. The composition of claim 1, wherein the vector is a non-viral vector.

32. The composition of claim 1, wherein the vector is an adenoviral vector or an adeno-associated viral (AAV) vector.

33. The composition of claim 1, wherein the vector is a plasmid.

34. The composition of claim 1, wherein the first promoter is CBH and the second promoter is selected from EFS or JeT.

35. The composition of claim 1, wherein the first promoter is CMV and the second promoter is selected from PGK, JeT, or EF1-α.

36. The composition of claim 1, wherein the modified GlcNAc-1 phosphotransferase comprises S1S3 PTase.

37. The composition of claim 1, wherein the lysosomal enzyme is selected from the group consisting of b-glucocebrosidase (GBA), Galactosylceremidase (GALC), a-Galactosidase (GLA), a-N-acetylglucosaminidase (NAGLU), acid a-glucosidase (GAA), lysosomal acid a-mannosidase (LAMAN), and HexM.

38. A method of treating a lysosomal storage disorder (LSD), the method comprising administering to a subject an effective amount of a composition of claim 1, wherein the composition increases the phosphorylation of a lysosomal enzyme responsible of the LSD, thereby treating the LSD.

39. The method of claim 10, wherein the subject has been diagnosed with the LSD.

40. The method of claim 10, wherein the subject presents a sign or symptom of the LSD.

41. A method of preventing an occurrence or an onset of a lysosomal storage disorder (LSD), the method comprising administering to a subject an effective amount of a composition of claim 1, wherein the composition increases the phosphorylation of a lysosomal enzyme responsible of the LSD, thereby preventing the occurrence of the LSD in the subject.

42. The method of claim 13, wherein the subject is at risk of the occurrence or the onset of the LSD.

43. The method of claim 13, wherein the subject presents a sign or a symptom of the LSD.

44. A method of ameliorating the phosphorylation of a lysosomal enzyme responsible for a lysosomal storage disorder (LSD), the method comprising contacting to a cell, an effective amount of a composition of claim 1, wherein the composition increases the phosphorylation of the lysosomal enzyme.

45. The method of claim 16, wherein the cell is in vitro or ex vivo.

46. The method of claim 16, wherein a subject comprises the cell.

47. The method of claim 18, wherein the subject presents a sign or a symptom of the LSD.

48. The method of claim 18, wherein the subject is at risk of the occurrence or the onset of the LSD.
